(19) United States
(12) Patent Application Publication
(10) Pub. No.: US 2013/0096437 A1
(43) Pub. Date: Apr. 18, 2013

(54) METHOD FOR DETECTING TEMPLE HOT SPOT TEMPERATURE OF A LIVE BODY

(71) Applicant: Radiant Innovation Inc., Hsinchu (TW)

(72) Inventor: VINCENT WENG, HSINCHU (TW)

(73) Assignee: RADIANT INNOVATION INC., HSINCHU (TW)

(21) Appl. No.: 13/711,770

(22) Filed: Dec. 12, 2012

Related U.S. Application Data
(63) Continuation-in-part of application No. 12/189,986, filed on Aug. 12, 2008.

(30) Foreign Application Priority Data
Aug. 21, 2007 (TW) 96130830

(51) Int. Cl. A61B 5/00; A61B 5/01

(52) U.S. Cl. CPC A61B 5/0075; A61B 5/01
USPC 600/474

(57) ABSTRACT

The present invention discloses a method for detecting temple hot spot temperature of a live body. Firstly, an infrared thermometer is used to continuously scan a temple of testee's forehead to obtain a plurality of measured infrared signals. Next, the measured infrared signals are sequenced from a greatest to a smallest measured infrared signal. Two output infrared signals closest to the greatest measured infrared signal are selected from the sequenced infrared signals. An average signal value is worked out from the selected infrared signals. Then, the average signal value is converted into a temple hot spot temperature of the testee's forehead. Thereby, not only the test result is more precise, but also the testee can learn the test result sooner.

### RELATED APPLICATIONS

The present invention is a continuation-in-part application of the application entitled “METHOD FOR DETECTING BODY TEMPERATURE OF LIVE BODY” (U.S. Ser. No. 12/189,986, filed 12 Aug. 2008, currently pending), and which is used herein for reference in its entirety.

### BACKGROUND OF THE INVENTION

1. Field of the Invention

The present invention relates to a method for detecting body temperature of a live body, particularly to a method for fast detecting temple hot spot temperature of a live body.

2. Description of the Related Art

Temperature is an important physiological index indicating whether a living body is healthy. The instruments for temperature measurement can be categorized into the contact thermometers and the non-contact thermometers. The contact thermometers include mercury thermometers and electronic thermometers. The non-contact thermometers include infrared thermometers, such as infrared ear thermometers, infrared forehead thermometers, and infrared ear-forehead thermometers. The infrared thermometers are getting more and more popular because they are easy to operate and prompt to output results.

The infrared ear thermometer is not always suitable to the persons having some ear defect or sleeping children. Contrarily, the infrared forehead thermometer can work in the abovementioned cases. A U.S. Pat. No. 6,292,685 disclosed an infrared forehead thermometer, wherein the probe of the infrared thermometer is used to scan the testee's forehead to obtain a plurality of measurement values, and the greatest one of the measurement values is converted into a forehead temperature. However, the greatest measurement value usually contains noise. Thus, the test result is likely inaccurate. Besides, the conventional infrared forehead thermometer has to convert each measured infrared signal into a forehead temperature immediately. Thus, the scanning speed thereof is reduced, and the accuracy thereof is decreased.

Accordingly, the present invention proposes another method for detecting body temperature of a live body to overcome the abovementioned problems.

### SUMMARY OF THE INVENTION

One objective of the present invention is to provide a method for detecting temple hot spot temperature of a live body, which firstly obtains an average infrared signal from several infrared signals and converts the average infrared signal into a temple hot spot temperature, and which outperforms the conventional technology that has to one by one convert infrared signals into temperature values and is hard to fast detect body temperature.

Another objective of the present invention is to provide a method for detecting temple hot spot temperature of a live body, which can fast obtain a high-accuracy result in temperature measurement.

The method for detecting temple hot spot temperature of a live body of the present invention comprises steps: using an infrared thermometer and continuously moving the infrared thermometer to scan different positions on a temple of testee's forehead to obtain a plurality of measured infrared signals; sequencing the plurality of measured infrared signals from a greatest measured infrared signal to a smallest measured infrared signal; selecting two output infrared signals, which are closest to the greatest measured infrared signal; obtaining an average signal value of the selected two output infrared signals; and converting the average signal value into a temple hot spot temperature of the testee's forehead.

In one embodiment of the present invention, the infrared thermometer scans the testee's forehead five times every second.

In one embodiment of the present invention, the infrared thermometer spends at least five seconds every time when scanning the testee's forehead.

Below, the technical contents of the present invention will be described in detail to enable the persons skilled in the art to easily understand the present invention.

It to be noted that the foregoing general description and the following detailed description are only intended to exemplify the present invention but not to limit the scope of the present invention.

### BRIEF DESCRIPTION OF THE DRAWINGS

FIG. 1 is a perspective view schematically showing an infrared forehead thermometer used in the description of the present invention;

FIG. 2 is a block diagram schematically showing the circuit of an infrared forehead thermometer used in the description of the present invention;

FIG. 3 is a flowchart of a method for detecting temple hot spot temperature of a live body according to the present invention; and

FIG. 4 is a diagram schematically showing the sequencing of infrared signals according to the present invention.

FIG. 5 is a diagram schematically showing the selecting two output infrared signals which are closest to the greatest measured infrared signal according to the present invention.

### DETAILED DESCRIPTION OF THE INVENTION

The present invention proposes a method for detecting temple hot spot temperature of a live body, which uses an infrared thermometer to fast detect a temple hot spot temperature of a testee's forehead and obtain a more precise temple hot spot temperature. Below, an infrared forehead thermometer is used as a measurement instrument to describe the technical contents of the present invention.

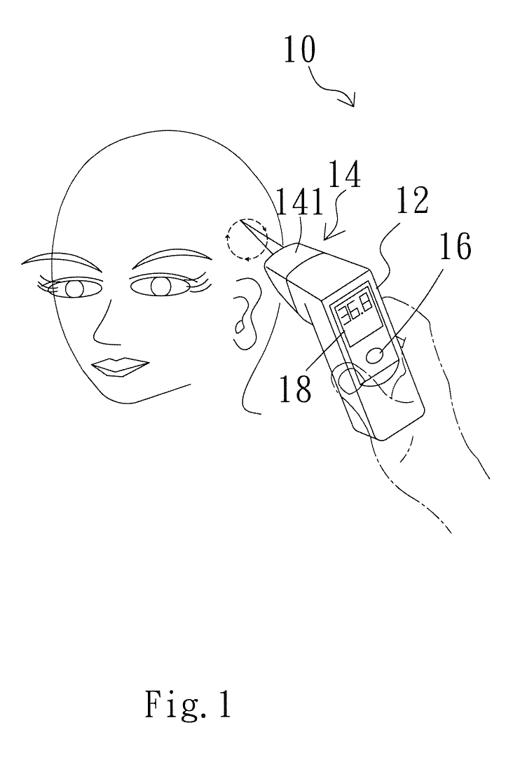

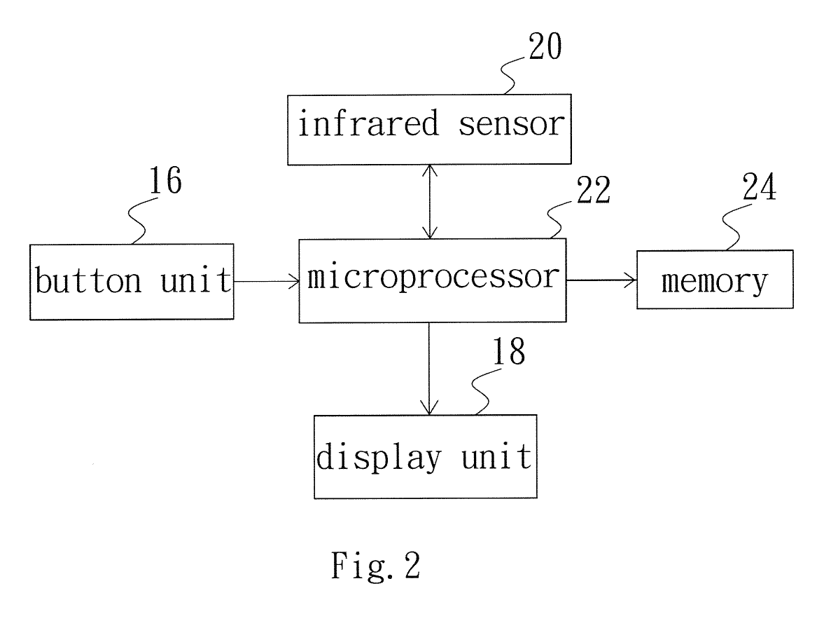

Refer to FIG. 1 and FIG. 2. FIG. 1 is a perspective view schematically showing an infrared forehead thermometer used in the description of the method of the present invention, and FIG. 2 is a block diagram schematically showing the circuit of the infrared forehead thermometer. The infrared forehead thermometer 10 comprises a body 12 and a probe structure 14 at one end of the body 12. The body 12 has a button unit 16 for operation and a display unit 18 presenting the results of measurement, such as a liquid crystal display. The probe structure 14 has a casing 141, an infrared-permeable window at the front end of the casing 141, and an infrared sensor 20 inside the casing 141. The infrared sensor 20 can detect the infrared radiation passing through the window. The infrared forehead thermometer 10 also comprises a microprocessor 22 and a memory 24 both arranged inside the body 12. The microprocessor 22 is coupled to the button unit 16, the display unit 18, the infrared sensor 20 and the memory 24 and controls the operation of the abovementioned components. The measurement results and other data are stored in the memory 24.

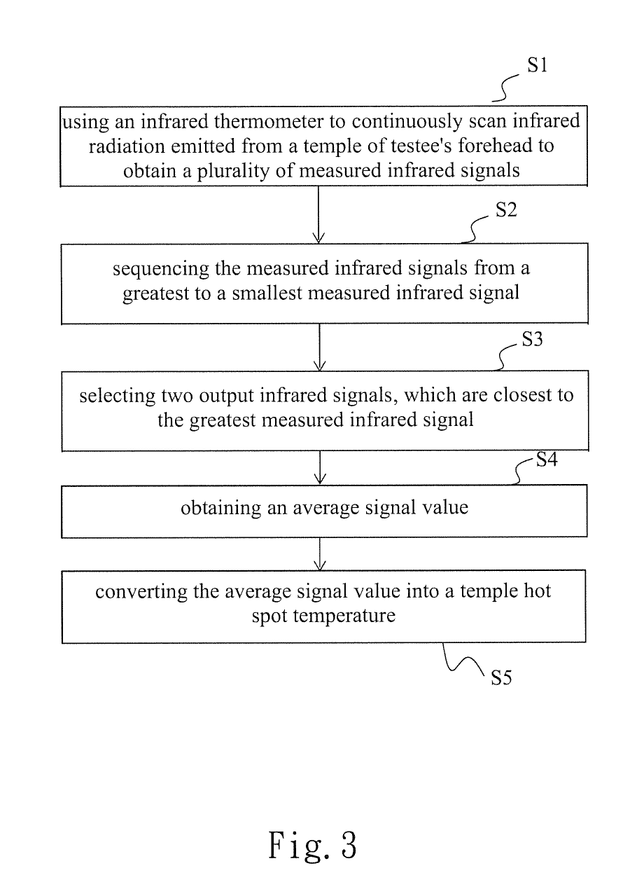

Refer to FIG. 3, which is a flowchart of a method for detecting temple hot spot temperature of a live body according to the present invention. In Step S1, the probe structure 14 of the infrared thermometer 10 is aimed at the temple of a testee's forehead and then gradually moved so that the infrared sensor 20 continuously scans and detects the infrared radiation emitted from the temple of the testee's forehead and then obtains a plurality of measured infrared signals.

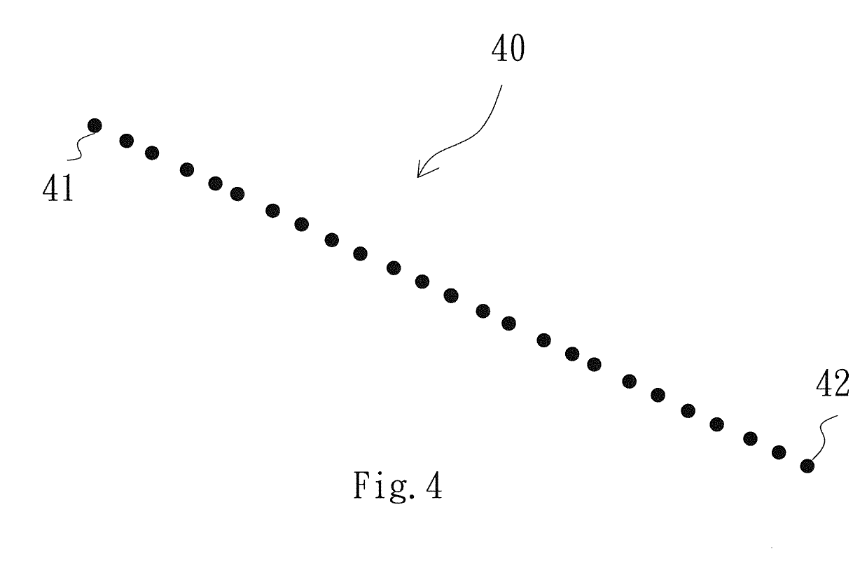

According to one embodiment of the present invention, the infrared thermometer 10 scans the testee's forehead five times every second. And, the infrared thermometer 10 spends at least five seconds every time when scanning the testee's forehead. That is to say, according to one embodiment of the present invention, a number of the plurality of measured infrared signals the infrared thermometer 10 scans should be no less than twenty-five, as shown in FIG. 4.

Next, in Step S2, the infrared sensor 20 transmits the plurality of measured infrared signals to the microprocessor 22, and the microprocessor 22 sequences the measured infrared signals from small to great or from great to small to identify a greatest measured infrared signal 41 and a smallest measured infrared signal 42, as shown in FIG. 4. According to an embodiment of the present invention, in order to obtain a precise temple hot spot forehead temperature, the microprocessor 22 may discard the greatest measured infrared signal 41 and the smallest measured infrared signal 42 and only output a plurality of output infrared signals 40.

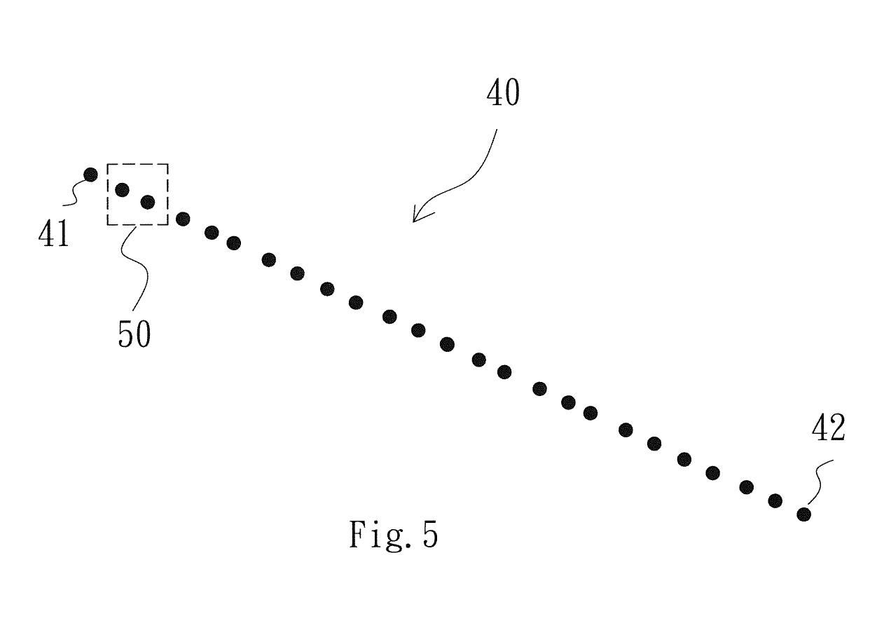

Next, in Step S3, as referring to FIG. 5, the microprocessor 22 selects two output infrared signals 50 which are closest to the greatest measured infrared signal 41 for further analysis. Hereinafter, the microprocessor 22 selects two output infrared signals 50 closest to the greatest measured infrared signal 41 to compute into a temple hot spot temperature of the testee's forehead.

Next, in Step S4, the microprocessor 22 works out and obtains an average signal value of the selected two output infrared signals. Generally speaking, the more numbers of output infrared signals are being selected, the more accurate a temple hot spot temperature can be reached.

Finally, in Step S5, the microprocessor 22 converts the average signal value into a temple hot spot temperature. Then, the microprocessor 22 transmits the temple hot spot temperature to the display unit 18, and the user can learn the measurement result from the display unit 18. The infrared thermometer 10 should detect the temperature related to the primary artery in the head. As shown in FIG. 1, the infrared thermometer 10 is continuously scanning the temple area because the artery in the temple interconnects with the heart via the carotid artery.

In conclusion, the method for detecting temple hot spot temperature of a live body of the present invention sequences the measured infrared signals and selects two appropriate infrared signals from the sequenced infrared signals which are closest to the greatest measured infrared signal for computing into a testee's temple hot spot temperature. The proposed method works out and obtains an average signal value from the selected infrared signals and converts the average signal value into the temple hot spot temperature of the testee's forehead. By employing the present method which continuously moves the infrared thermometer and continuously scans different positions on the temple of the testee's forehead, the present invention can solve the problem of the conventional technology that has to convert all infrared signals into temperatures one by one and is hard to fast obtain a precise measurement result.

Since the conventional technology adopts the highest temperature as the measurement output, the highest temperature is worked out from the greatest infrared signal, and the greatest infrared signal contains a notable proportion of noise, the present invention utilizes only several infrared signals (i.e. two) closest to the greatest infrared signal. Therefore, the present invention has a more precise measurement result.

The embodiments described above are to demonstrate the technical contents and characteristics of the present invention and to enable the persons skilled in the art to understand, make, and use the present invention. However, it is not intended to limit the scope of the present invention. Therefore, any equivalent modification or variation according to the spirit of the present invention is to be also included within the scope of the present invention.

What is claimed is:

1. A method for detecting temple hot spot temperature of a live body comprising steps:
using an infrared thermometer and continuously moving said infrared thermometer to scan different positions on a temple of testee's forehead to obtain a plurality of measured infrared signals;
sequencing said plurality of measured infrared signals from a greatest measured infrared signal to a smallest measured infrared signal;
selecting two output infrared signals, which are closest to said greatest measured infrared signal;
obtaining an average signal value of said selected two output infrared signals; and
converting said average signal value into a hot spot temperature of said temple of testee's forehead.

2. The method for detecting temple hot spot temperature of a live body according to claim 1, wherein said infrared thermometer scans said testee's forehead five times every second.

3. The method for detecting temple hot spot temperature of a live body according to claim 2, wherein said infrared thermometer spends at least five seconds when scanning said testee's forehead.

4. The method for detecting temple hot spot temperature of a live body according to claim 1, wherein said infrared thermometer has an infrared sensor detecting infrared radiation emitted by said testee's forehead.

5. The method for detecting temple hot spot temperature of a live body according to claim 4, wherein said infrared thermometer has a microprocessor; said microprocessor is coupled to said infrared sensor, receives said infrared signals from said infrared sensor, sequences said infrared signals, obtains said average signal value, and converts said average signal value into said hot spot temperature of said temple of testee's forehead.

6. The method for detecting temple hot spot temperature of a live body according to claim 4, wherein said infrared thermometer has a probe structure; said probe structure has a casing accommodating said infrared sensor; one front end of said casing has a window, and said infrared sensor receives infrared radiation passing through said window.
